(19) United States
(12) Patent Application Publication
(10) Pub. No.: US 2025/0302283 A1
(43) Pub. Date: Oct. 2, 2025

(54) COMPACT ROBOTIC ENDOSCOPE WITH BUILT-IN GRASPER

(71) Applicant: Xiaolong OUYANG, Bellevue, WA (US)

(72) Inventor: Xiaolong OUYANG, Bellevue, WA (US)

(21) Appl. No.: 18/622,471

(22) Filed: Mar. 29, 2024

Related U.S. Application Data
(63) Continuation-in-part of application No. 17/583,095, filed on Jan. 24, 2022, now Pat. No. 11,944,267.
(63) Continuation-in-part of application No. 18/083,209, filed on Dec. 16, 2022.
(60) Provisional application No. 63/460,728, filed on Apr. 20, 2023.
(60) Provisional application No. 63/461,939, filed on Apr. 26, 2023.
(60) Provisional application No. 63/462,647, filed on Apr. 28, 2023.
(60) Provisional application No. 63/462,985, filed on Apr. 29, 2023.
(60) Provisional application No. 63/464,571, filed on May 6, 2023.
(60) Provisional application No. 63/466,318, filed on May 14, 2023.
(60) Provisional application No. 63/535,077, filed on Aug. 29, 2023.
(60) Provisional application No. 63/544,497, filed on Oct. 17, 2023.
(60) Provisional application No. 63/544,645, filed on Oct. 18, 2023.
(60) Provisional application No. 63/544,789, filed on Oct. 19, 2023.
(60) Provisional application No. 63/544,963, filed on Oct. 20, 2023.
(60) Provisional application No. 63/599,991, filed on Nov. 16, 2023.
(60) Provisional application No. 63/602,405, filed on Nov. 23, 2023.
(60) Provisional application No. 63/608,316, filed on Dec. 11, 2023.
(60) Provisional application No. 63/613,772, filed on Dec. 22, 2023.
(60) Provisional application No. 63/620,838, filed on Jan. 14, 2024.
(60) Provisional application No. 63/623,236, filed on Jan. 20, 2024.
(60) Provisional application No. 63/623,410, filed on Jan. 22, 2024.
(60) Provisional application No. 63/623,418, filed on Jan. 22, 2024.
(60) Provisional application No. 63/624,086, filed on Jan. 23, 2024.
(60) Provisional application No. 63/554,976, filed on Feb. 17, 2024.
(60) Provisional application No. 63/555,552, filed on Feb. 20, 2024.
(60) Provisional application No. 63/556,151, filed on Feb. 21, 2024.
(60) Provisional application No. 63/556,151, filed on Feb. 21, 2024.
(60) Provisional application No. 63/556,712, filed on Feb. 22, 2024.
(60) Provisional application No. 63/562,687, filed on Mar. 7, 2024.

(51) Int. Cl. A61B 1/00

(52) U.S. Cl. CPC A61B 1/00147; A61B 1/00087; A61B 1/00103

(57) ABSTRACT

A medical endoscope that has a single-use portion with a built-in surgical implement such as a grasper and physical indicia that immediately and clearly show how far a distal end of the implement has protruded from a distal end of a cannula. The indicia can be three dimensional to give a tactical indication in addition to a visual indication of how far the surgical device protrudes from the cannula.

### REFERENCE TO RELATED APPLICATIONS

This application is a continuation in-part of each of (a) U.S. patent application Ser. No. 17/583,095 filed Jan. 24, 2022 (scheduled to issue Apr. 2, 2024 as U.S. Pat. No. 11,944,267) and (b) U.S. patent application Ser. No. 18/083,209 filed Dec. 12, 2022.

This application claims priority to U.S. Provisional Patent Applications: 63/454,640, filed Mar. 25, 2023, 63/454,640, filed Mar. 25, 2023, 63/454,953, filed Mar. 27, 2023, 63/458,178, filed Jan. 11, 2023, 63/460,728, filed Apr. 20, 2023, 63/461,939, filed Apr. 26, 2023, 63/462,647, filed Apr. 28, 2023, 63/462,985, filed Apr. 29, 2023, 63/464,571, filed May 6, 2023, 63/466,318, filed May 14, 2023, 63/535,077, filed Aug. 29, 2023, 63/544,645, filed Nov. 13, 2023, 63/544,497, filed Oct. 17, 2023, 63/544,789, filed Oct. 18, 2023, 63/544,963, filed Oct. 20, 2023, 63/554,976, filed Feb. 17, 2024, 63/556,151 filed Feb. 20, 2024, 63/556,712, filed Feb. 22, 2024, 63/599,991, filed Nov. 21, 2023, 63/602,405, filed Nov. 23, 2023, 63/608,316, filed Dec. 11, 2023, 63/613,772, filed Dec. 12, 2023, 63/620,838, filed Jan. 14, 2024, 63/623,236, filed Jan. 20, 2024, 63/623,410, filed Jan. 22, 2024, 63/623,418, filed Jan. 22, 2024, 63/624,086, filed Jan. 23, 2024, 63/554,976, filed Feb. 17, 2024, 63/555,552, filed Feb. 20, 2024, 63/556,151, filed Feb. 21, 2024, 63/556,712, filed Feb. 22, 2024, 63/562,687, filed Mar. 7, 2024, 63/176,307, filed Apr. 18, 2021, 63/285,061, filed Dec. 1, 2021, 63/295,913, filed Jan. 2, 2022, 63/299,829, Jan. 14, 2022, 63/302,563, filed Jan. 25, 2022, and 63/303,690, filed Jan. 27, 2022, 63/310,336, filed Feb. 15, 2022,

This application incorporates by reference the entirety of the foregoing non-provisional and provisional patent applications and claims the benefit of the filing date of each as well as of the applications that they incorporated by reference, directly or indirectly, and the benefit of which they claim, including U.S. provisional applications, U.S. non-provisional applications, and international applications.

This patent application incorporates by reference each of the following U.S. patents and U.S. and international (PCT) patent applications:
Ser. No. 16/972,989 filed Dec. 7, 2020;
PCT/US21/50095 filed Sep. 13, 2021;
PCT/US2017/053171, filed Sep. 25, 2017
Ser. No. 17/835,624 filed Jun. 8, 2022;
Ser. No. 16/363,209, filed Sep. 25, 2017, now U.S. Pat. No. 11,832,797;
Ser. No. 17/843,217, filed Jun. 17, 2022
PCT/US16/18670 filed Feb. 19, 2016;
Ser. No. 14/913,867 filed Feb. 23, 2016, now U.S. Pat. No. 10,874,287;
PCT/US16/65396 filed Dec. 7, 2016;
Ser. No. 15/371,858 filed Feb. 20, 2018, now U.S. Pat. No. 9,895,048;
Ser. No. 15/462,331 filed Mar. 17, 2017, now U.S. Pat. No. 10,524,636;
Ser. No. 15/651,526 filed Jul. 17, 2017, now U.S. Pat. No. 10,278,563;
Ser. No. 15/855,532 filed Dec. 27, 2017, now U.S. Pat. No. 10,292,571;
PCT/US18/14880 filed Jan. 23, 2018;
Ser. No. 16/407,028 filed May 8, 2019, now U.S. Pat. No. 11,253,141;
Ser. No. 16/413,160 filed May 15, 2019, now U.S. Pat. No. 10,869,592;
Ser. No. 16/447,251 filed Jun. 20, 2019, now U.S. Pat. No. 11,013,141;
PCT/US20/38349 filed Jun. 18, 2020;
PCT/US20/46018 filed Aug. 12, 2020;
Ser. No. 17/122,282 filed Dec. 15, 2020;
Ser. No. 17/145,466 filed Jan. 11, 2021, now U.S. Pat. No. 11,395,579;
Ser. No. 17/370,575 filed Jul. 8, 2021;
Ser. No. 17/349,674 filed Jun. 16, 2021;
Ser. No. 17/573,095 filed Jan. 24, 2022;
PCT/US2017/053171, filed Sep. 25, 2017
Ser. No. 17/835,624 filed Jun. 8, 2022;
Ser. No. 16/363,209, filed Sep. 25, 2017, now U.S. Pat. No. 11,832,797;
Ser. No. 17/843,217, filed Jun. 17, 2022;
Ser. No. 17/362,043, filed Jun. 29, 2021, now U.S. Pat. No. 11,350,816;
Ser. No. 17/473,587, filed Sep. 13, 2021, now U.S. Pat. No. 11,330,973;
Ser. No. 17/745,526, filed May 16, 2022;
Ser. No. 17/521,397, filed Nov. 8, 2021;
Ser. No. 17/720,143, filed Apr. 13, 2022;
Ser. No. 18/212,621, filed Jun. 21, 2023;
Ser. No. 17/941,884, filed Sep. 9, 2022;
Ser. No. 17/835,624, filed Jun. 8, 2022, now U.S. Pat. No. 11,684,248
Ser. No. 18/083,209, filed Dec. 16, 2022;
Ser. No. 18/113,395, filed Feb. 23, 2023;
Ser. No. 18/211,486, filed Jun. 19, 2023;
Ser. No. 18/233,282, filed Aug. 11, 2023;
Ser. No. 18/374,740, filed Sep. 29, 2023;
Ser. No. 14/913,867, filed Feb. 23, 2016, now U.S. Pat. No. 10,874,287;
Ser. No. 16/407,028, filed May 18, 2019, now U.S. Pat. No. 11,253,141;
Ser. No. 16/413,160, filed May 15, 2019, now U.S. Pat. No. 10,869,592;
Ser. No. 17/122,282, filed Dec. 15, 2020, now U.S. Pat. No. 11,844,498;
Ser. No. 15/371,858, filed Dec. 7, 2016, now U.S. Pat. No. 9,895,048;
Ser. No. 13/094,415, filed Apr. 26, 2011, now U.S. Pat. No. 9,649,014;
Ser. No. 15/462,331, filed Mar. 17, 2017, now U.S. Pat. No. 10,524,636;
Ser. No. 15/651,526, filed Jul. 17, 2017, now U.S. Pat. No. 10,278,563;
Ser. No. 15/855,532, filed Dec. 27, 2017, now U.S. Pat. No. 10,292,571;
Ser. No. 15/484,953, filed Apr. 11, 2017, now U.S. Pat. No. 10,426,320;
PCT/US2020/038349, filed Jun. 18, 2020; now U.S. Pat. No. 11,013,396;
Ser. No. 17/370,575, filed Jul. 8, 2021;
Ser. No. 18/209,947, filed Jun. 14, 2023;
Ser. No. 17/145,466, filed Jan. 11, 2021, now U.S. Pat. No. 11,395,579;
Ser. No. 16/664,082, filed Oct. 25, 2019, now U.S. Pat. No. 11,071,442;
Ser. No. 17/349,674, filed Jun. 16, 2021
PCT/US2020/046018, filed Aug. 12, 2020;
Ser. No. 17/583,095, filed Jan. 24, 2022;
Ser. No. 13/276,839, Oct. 19, 2011, now U.S. Pat. No. 8,702,594; and
Ser. No. 13/094,415, Apr. 26, 2011, now U.S. Pat. No. 9,649,014.

### FIELD

This patent specification relates to endoscopy instruments and methods. Some embodiments relate to portable endoscopy instruments that include a reusable portion and a releasably attached single-use portion and some embodiments relate to such endoscopy instruments that include a grasper or another surgical implement.

### BACKGROUND

Medical practitioners such as urologists and gynecologists have long relied on endoscopes to view and treat internal tissue. In the case of both rigid and flexible conventional endoscopes, the optical system and related components are relatively expensive and are intended to be re-used many times. They require stringent decontamination and disinfection procedures after each use, which adds significant expense in equipment and labor. In recent years, disposable or partly endoscopes have been developed and improved. They typically comprise a single-use portion that includes a cannula with an imaging module at the distal end. The single-use portion releasably attaches to a reusable portion that includes image processing electronics and a display. Alternatively, the reusable portion may have limited electronics, or none, and may carry no display. In that case, an imaging module at the tip of the cannula is coupled with a remote processing unit and/or a remote display operatively coupled with the imaging module wirelessly or via cables. Disposable or single-use endoscopy significantly lessens the risk of cross-contamination and hospital-acquired diseases and eliminates the expense and time for decontamination and disinfection. Disposable endoscopes find applications in medical procedures such as imaging and treating the male and female urinary system and the female reproductive system and other internal organs. Examples of disposable or single-use endoscopes are discussed in the patents and applications incorporated by reference herein.

When a surgical implement such as a grasper is to be introduced through a lumen or working channel in an endoscope, traditionally two practitioners or clinicians participate—one to hold and manipulate the endoscope and another to thread the surgical implement through a proximal port in the endoscope. Parent application Ser. No. 17/583,095, to issue as U.S. Pat. No. 11,944,267, describes an improvement in which a surgical instrument such as a grasper can be built in an endoscope, enabling a single practitioner to manipulate both the endoscope and the surgical implement. This patent application is directed to a further improvement related to use of surgical implements introduced through a lumen in an endoscope and especially a portable endoscope with a single-use cannula and related components.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

As described in the initially presented claims but subject to amendments thereof in prosecuting this patent application, according to some embodiments, a compact robotic endoscope comprises a reusable portion; a single-use portion comprising (a) a proximal part configured to releasably couple to the reusable portion to form said endoscope, (b) a hub that extend distally from the proximal part along the cannula axis and has a hub port, (c) a cannula that extends distally from the hub and has an imaging module and a distal port at a distal part thereof, and (d) an internal lumen that extends from the hub port to said distal port; a surgical instrument comprising a push-pull assembly that includes a finger-hold, a push-pull button configured to move distally and proximally relative to the finger-hold, and a cable with a surgical device secured to a distal end thereof; wherein a proximal end of said cable is secured to said push-pull button and the cable extends therefrom to and though said hub port and said lumen to said surgical device and is configured to move the surgical device distally and proximately relative to the cannula with motion of the push-pull button relative to the finger-hold; a locking knob secured to an intermediate portion of the cable and configured to move distally and proximally relative to the hub with said cable and to selectively lock the cable to the hub to thereby lock the surgical device at a selected position relative to the cannula; and readily discernable indicia structures on the hub and/or on the push-pull button configured to indicate an axial position of said locking knob relative to the hub and/or said push-pull button relative to the finger-hold and thus axial position of said surgical device relative to said cannula.

According to some embodiments the endoscope further comprises one or more of: (a) the cannula is configured to rotate about a cannula axis relative to said reusable portion, and an electric motor operatively coupled with the cannula to rotate the cannula about said cannula axis through selected angles relative to the reusable portion and a manual control operatively coupled with the motor to selectively cause the motor to rotate the cannula through said angles.; (b) the surgical device secured to the distal end of the cable comprises a grasper configured to move between a retracted position in the cannula and an extended position in which the grasper protrudes distally from the cannula; (c). the grasper is spring-biased to open with distal axial motion relative to the cannula and to close with proximal axial motion relative to the cannula; (d) the distal part of the cannula is configured to angulate and said reusable portion includes a manual control to cause the cannula to angulate through selected angles relative to the long dimension of the cannula; (e). the reusable portion comprises a slot extending along a cannula axis and configured to enable insertion therein of said proximal part of the single-use portion in a motion transverse to the cannula axis; (f) .the cannula further includes a proximal fluid port coupled with said lumen and configured to selectively rotate with the cannula about said cannula axis relative to the reusable portion; (g) .the hub port is valved to prevent fluid flow out of the hub port; (h) the surgical device secured to the distal end of the cable comprises a grasper having distal resilient jaws spring-biased away from each other and proximal arch-shaped portions configured to engage said cannula as the grasper moves proximally relative to the cannula and thereby overcome said spring-bias and bring the jaws closer to each other; (i) .the single-use portion together with said surgical instrument is configured for shipment to an end user in a sterile package; and (j) .the surgical instrument is permanently built into said single-use portion.

According to some embodiments, an endoscope comprises: a reusable portion and a single-use portion releasably secured to the reusable portion; wherein the single-use portion comprises a proximal part configured to releasably couple to the reusable portion to form said endoscope, a hub extending distally from the proximal part along a cannula axis, a cannula extending distally from the hub, and an internal lumen; wherein: the cannula has at a distal part thereof an imaging module and a distal port communicating with said lumen; the hub has a hub port communicating with said lumen; and the single use portion further includes a surgical instrument comprising a manually operated control assembly secured to the hub port, a cable that passes through the hub port and the lumen, and a surgical device at a distal end of the cable and configured to move relative to the cannula between a distal position and a retracted position in response to manual operation of said control assembly; and physical indicia on at least one of the hub and the control assembly moving relative to said hub and/or control assembly in a motion matching said motion of the surgical device relative to the cannula and providing an indication of the instantaneous position of the surgical device relative to the cannula.

According to some embodiments, the endoscope described in the immediately preceding paragraph further comprises one or more of: (a) the endoscope further includes a locking mechanism secured to at least one of the cable and the control assembly and configured to selectively lock said surgical device in a selected position relative to the cannula; (b) the indicia comprise indentations of ridges on the hub and said locking mechanism comprises a knob secured to said cable and configured to move therewith relative to said indentations and thereby instantaneously indicate the position of the surgical device relative to the cannula; and (c) the manually operated control assembly comprises a finger-hold configured to maintain a set distance from the hub port and a push-pull element coupled to a proximal end of the cable and configured to move relative to said finger-hold, and said indicia comprise indentations of ridges on a the movable element that mover relative to the finger-hold to thereby indicate the position of the surgical device relative to the cannula.

According to some embodiments, an endoscopy method comprises: introducing a cannula portion of a single-use portion of the endoscope into an interior space in a body; taking images of the interior space with an imaging module at a tip of the cannula; manually operating a control assembly built in the single-use portion to selectively move a cable that originates at the control assembly and passes through a proximal port in the single-use portion and a lumen therein and terminates distally in a surgical device that moves between a distal position in which at least a portion thereof protrudes from the cannula and a retracted position in the cannula; and detecting the position of the surgical device relative to the cannula by detecting visible displacement of the cable or of an element secured thereto relative to physical indicia that are on said single-use portion and are proximal of the cannula.

According to some embodiments, the endoscopy method further comprises: (a) the step of manually operating the control assembly comprises selectively moving a push-pull button relative to a finger-hold to thereby push or pull said cable axially along a length of said lumen and thereby move said surgical device distally or proximally relative to a distal end of the cannula; (b) the step of moving the surgical device relative to the cannula comprises moving a grasper that has distal jaws biased away from each other and configured to move away from each other as the grasper moves distally relative to the cannula and to move toward each other as the grasper moves proximally relative to the cannula; (c), selectively locking the surgical device in a selected position relative the cannula using a locking knob coupled to the cable and configured to selectively lock the cable to the cannula; and (d) .the step of detecting the position of the surgical device relative to the cannula comprises detecting the position of said locking knob relative to said indicia on said single-use portion.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail using the accompanying drawings in which:

FIG. 1 is a perspective view of an assembled endoscope with an integrated surgical implement, according to some embodiments.

FIG. 2 is a perspective view of a single-use portion of an endoscope from a right-side viewpoint, according to some embodiments.

FIG. 3 is a perspective view of a single-use portion of an endoscope from another right-side viewpoint, according to some embodiments.

FIG. 4 is a perspective view of a single-use portion of an endoscope from a top-side viewpoint, according to some embodiments.

FIGS. 5A-5D are partly sectional and partly side elevational views of a distal part of a cannula with a grasper in different positions relative to the cannula, according to some embodiments.

FIG. 6 is a perspective exploded view of an endoscope illustrating a reusable portion of an endoscope and how a single-use portion mounts thereto, according to some embodiments.

### DETAILED DESCRIPTION

A detailed description of examples of preferred embodiments is provided below. While several embodiments are described, the new subject matter described in this patent specification is not limited to any one embodiment or combinations of embodiments described herein but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features.

Like reference numbers and designations in the various drawings indicate like elements. Further, the reference numbers of components that are like in structure and function have the same second and third digits. For conciseness, components that bear the same reference numbers of the same last two digits of reference numbers are described only in connection with the Figure that first refers to them and the description is not repeated in connection with subsequently discussed Figures.

When a surgical implement such as a grasper is introduced into a patient through a lumen or working channel in an endoscope, the practitioner carrying out the medical procedure typically relies on visualizing the distal end of the instrument in images from an imaging module at the tip of a cannula. However, the view may not be sufficiently clear, or a practitioner may not be looking at the time at a display of such views. In such cases, a practitioner may additionally rely on experience and feel to assess how far the surgical instrument has protruded distally from the cannula. It may be possible in some cases to observe the cannula tip and the tip of a surgical instrument using another modality such as ultrasound or x-ray imaging or another kind of imaging, but this may not be practicable in many cases.

In addition to providing a visual indication of how far a surgical device such as a grasper has protruded from the cannula and, in the case of a grasper in the form described below, how fac the grasper has opened or closed, the physical indicia can be made sufficiently prominent to provide a tactile indication. For example, the indicia can be ridges that are big enough to be felt by touch, even with a gloved finger, that that a practitioner can tell by touch how far a surgical device extends distally from a cannula and/or how open or closed a grasper is.

As described in detail below, the subject patent application discloses a particularly convenient and efficacious way to improve the use of surgical implements such as a grasper with endoscopes and particularly endoscopes with a single-use cannula and related components.

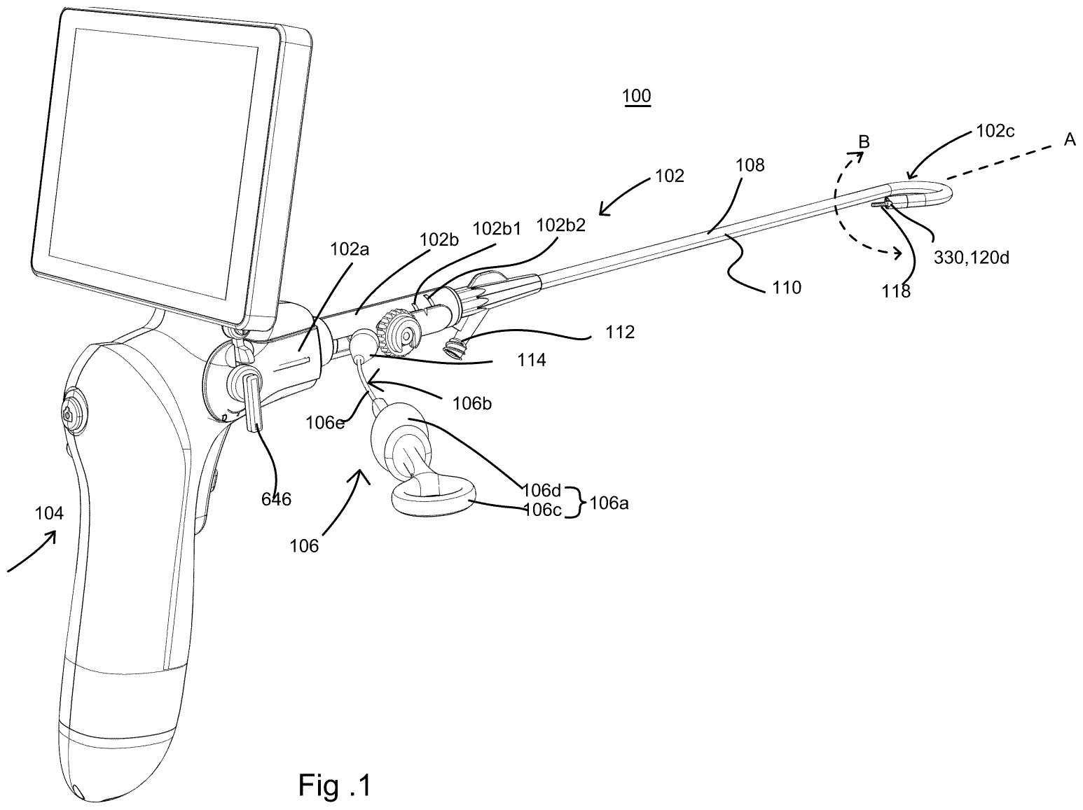

FIG. 1 is a perspective view of an assembled endoscope 100 with an integrated surgical implement from a right-side viewpoint, according to some embodiments. The Figure illustrates endoscope 100 in an assembled state, with a single-use portion 102 releasably secured to a reusable portion 104 and a surgical implement 106 secured to single-use portion 102.

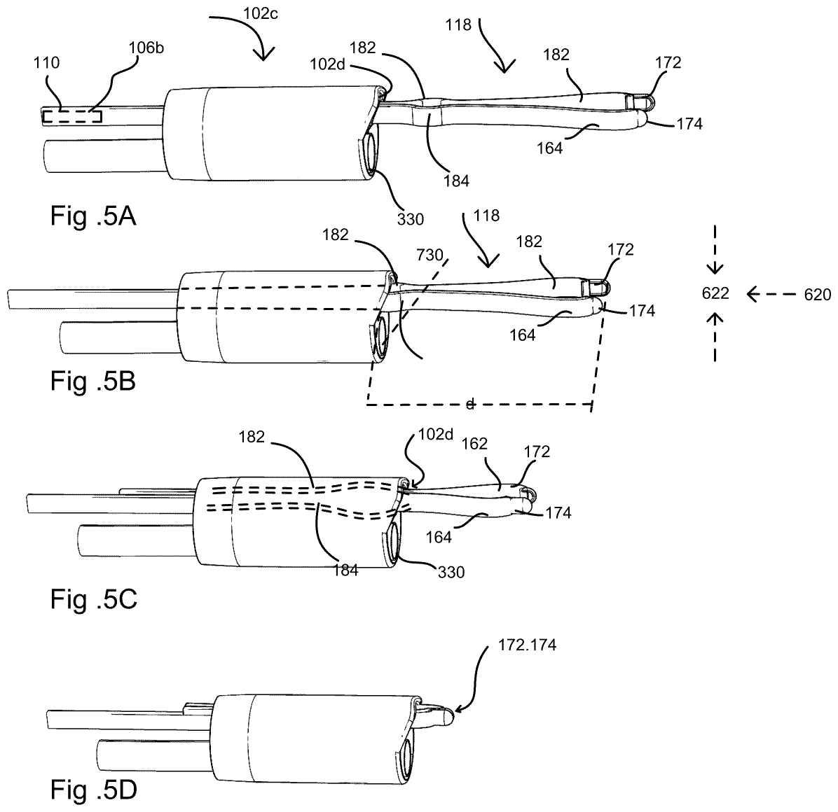

Single-use portion 102 comprises (a) a proximal part 102a configured to releasably couple to reusable portion 104, (b) a hub 102b that extend distally from proximal part 102a along a cannula axis A and has a fluid port 112 and a surgical implement port 114, (c) a cannula 108 that extends distally from hub 102b along cannula axis A, has a bendable distal part 102c tipped with an imaging module 330 and distal port 102d (FIG. 5A), and further has at least one internal channel or lumen 110 extending from ports 112 and 114 to distal port 102d. Ports 114 and 112 can connect to the same internal lumen 110 or to respective different internal lumens.

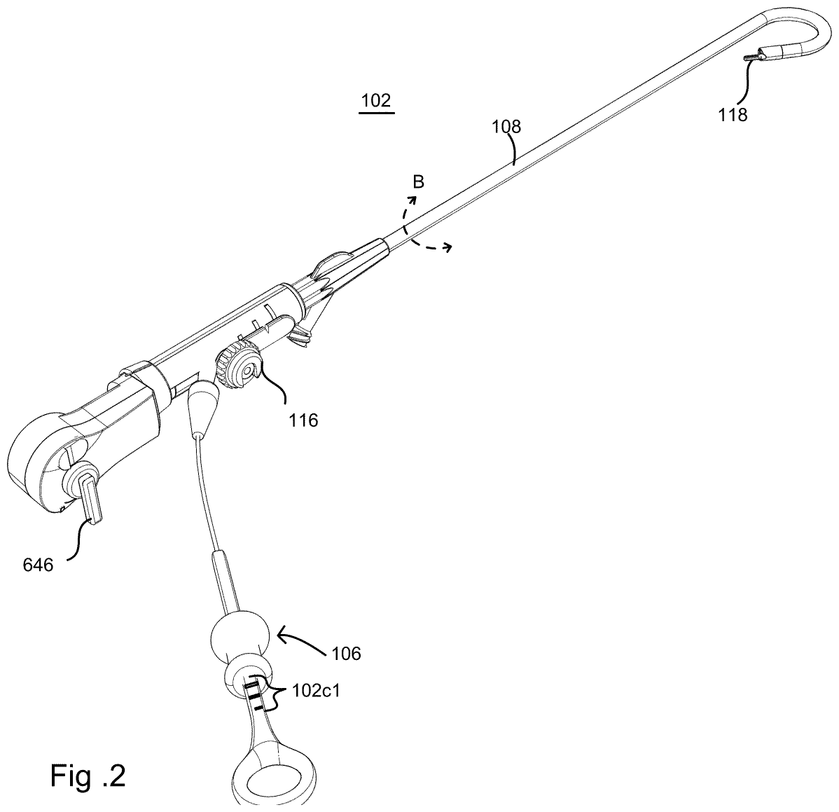

Surgical implement 106 comprises a manually operated control assembly that can be in the form of push-pull assembly 106a (FIGS. 1 and 2) and a cable 106b that extends from assembly 106a through port 114 and lumen 110 to or through distal port 102d and terminates distally at a device such as grasper 118. Assembly 106a comprises a push-pull button 106c to which a proximal end of cable 106b is secured, a finger-hold 106d in which a distal part of push-pull button 106c slides distally and proximally, a hollow conduit or casing 106e in which a portion of cable 106b between push-pull button 106c and port 114 slides distally and proximately. In some embodiments, conduit 106e can be omitted and a distal end of finger-hold 106d or an extension thereof can be directly secured to port 114. Conduit 106e has a distal end secured to port 114, for example by a Luer coupling. If conduit 106e is omitted, a distal end of finger-hold 106d or an extension thereof can similarly couple to port 114. Alternatively, different coupling methods can be used to secure conduit 106e or finger-hold 106d to port 114. A backflow prevention valve (not shown) can be used at port 114 to keep fluid from being expelled from port 114.

In operation, during insertion of cannula 108 in a patient push-pull button 106c is retracted proximally relative to finger-hold 106d such that a distal end of surgical implement 106, such as a grasper 118, is retracted or mostly retracted in cannula 102. When desired during a medical procedure in which cannula 102 is in the patient, a practitioner moves push-pull button 106c distally relative to finger-hold 106d to thereby push grasper 118 distally out of cannula 108. When desired, the practitioner pulls push-pull button 106c proximally relative to finger-hold 106d to thereby retract grasper 118 toward or into cannula 108. As described in more detail further below, such movement of grasper 118 relative to cannula 108 opens or closes jaws of the grasper accordingly.

Importantly, push-pull assembly 106a is configured to provide the practitioner with an immediate, clear awareness of the position of grasper 118 relative to cannula 108. To this end, hub 102b has indicia structures such as several indentations of ridges of which only two are numbered in FIG. 2 as indicia 102b1 and 102b2. The indicia are spaced from each other along cannula axis A, and locking mechanism, for example in the form of a knob 116 is secured to a portion of cable 106b that is inside hub 102b to move therewith along cannula axis A. The axial position of knob 116 relative to indicia such as 102b1 and 102b2 therefore corresponds to the axial position of grasper 118 relative to cannula 102. Indicia such as 102b1 and 102b2 are spaced axially from each other to correspond to displacement of grasper 118 relative to cannula 102 in some measures of distance, such as mm or some fraction of an axial length of grasper 118. Alternatively, or in addition, several comparable indicia structures 102c1 FIG. 2) are on push-pull button 106c to indicate the axial position of button 106c relative to finger-hold 102d and thus the position of grasper 118 relative to distal end 102c of cannula 102.

As noted above, the indicia can be made sufficiently large to provide a tactile indication in addition to a visual indication, for example by making the indicial grooves or ridges large enough to be felt with a finger, even a gloved finger.

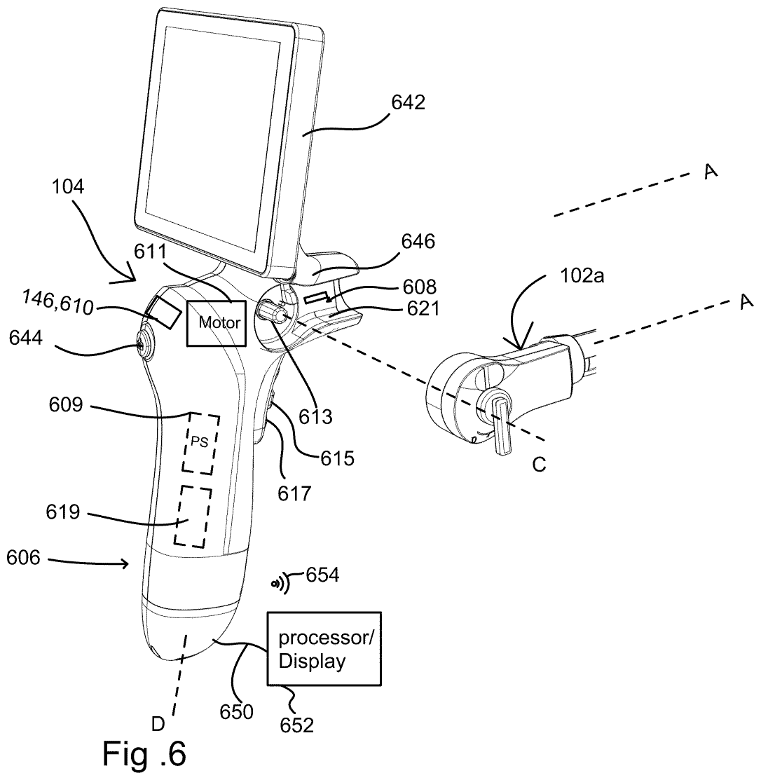

An example of a grasper such as grasper 118 is described in detail in said parent application Ser. No. 17/583,095 scheduled to issue as U.S. Pat. No. 11,944,267, which is incorporated by reference herein. FIGS. 5A-5D herein are like FIGS. 6A-6D respectively in said parent application except that the FIG. numbers and some reference numerals have been conformed to the nomenclature used in this patent specification.

FIGS. 5A-5D are partly sectional and partly side elevational views of distal part 102c of cannula 102, with grasper 118 in different positions relative to the cannula, according to some embodiments. FIG. 5A shows grasper 118 and grasper jaw portions 162 and 164 and claw portions 172 and 174 in an open position. In this position, arch shaped portions 182 and 184 of grasper 118 protrude distally from distal port 102d at distal part 102c of cannula 102. Grasper 118 is configured to be spring biased toward keeping its jaws 172 and 174 apart. While grasper 118 is in a distally protruded position from distal port 102d of cannula 102 as shown in FIG. 5A, jaw portions 162 and 164 and claw portions 172 and 174 are apart from each other.

FIG. 5B shows grasper 118 after cable 106b has pulled it proximally some distance relative to cannula 102. Now grasper 118 has moved proximally relative to distal port 102d over a part of its axial length, in the direction indicated by dashed arrow 620. A practitioner accomplishes this by manually pulling push-pull button 106c distally relative to finger-hold 106d. In the position shown in FIG. 5B, the proximal ends of arch shaped portions 182 and 184 are just beginning to engage with a distal edge of distal port 102d. This engagement forces claw portions 172 and 174 towards each other, as shown by dashed arrows 622 in FIG. 5B. Due to the distance d between the distal port 102d and claw portions 172 and 174, a camera module 330 having a wide-field of view (depicted by lines 730) should have a good view of claw portions 172 and 174 and any tissue or object that they may hold or target (not shown).

FIG. 5C shows grasper 118 after it has moved further proximally relative to cannula distal port 102d of cannula 102 and grasper claws 172 and 174 are clamped against each other. As seen in FIG. 5C, arch portions 182 and 184 are shown retracted proximally of distal port 102d and have been forced toward each other by contact with inner structures of lumen 110 that are just proximal to distal port 102d. This forcing of arch portions 182 and 184 toward each other in turn forces claw portions 172 and 174 to a closed position to hold any tissue or other object that might be grasped thereby (not shown).

FIG. 5D shows grasper 118 after it has been retracted even more proximally than in FIG. 5C. In FIG. 5D, claw portions 182 and 184 are shown nearly flush with a distal face of distal port 102d.

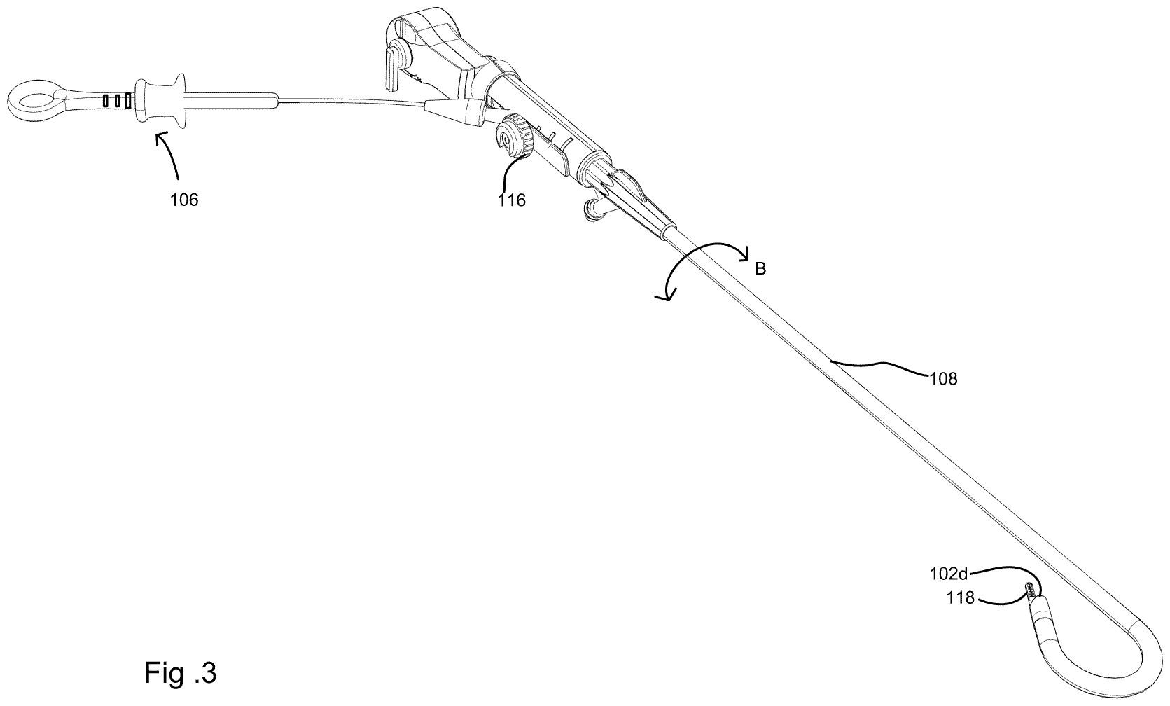

Referring back to FIGS. 1-3, the motion of grasper 118 relative to distal end 102c of cannula 102 is due to a practitioner manually moving push-pull button 106c relative to finger-hold 106d. As grasper 118 moves relative to distal port 102d, locking knob 116 correspondingly moves axially relative to hub 102b and relative to indicia structures thereon such as indentations or ridges 102b1, 102b2, etc. Push-pull button 106c moves axially correspondingly relative to finger-hold 106d and thereby several indicia structures 106C1 (if used in an example of endoscope 100) similarly show the axial position of grasper 118 relative to distal port 102d of cannula 102. This can keep a practitioner immediately aware of the position of grasper 118 relative to cannula 102 and the positions of grasper jaws 172 and 174 relative to each other even if a view of grasper 118 in images from imaging module 330 is not entirely clear or easy to interpret and even if the practitioner is not at the time looking at images from imaging module 330.

Locking knob 116 can lock cable 106b in place relative to hub and thus to lock grasper 118 in a selected position relative to cannula 102. Knob 116 can rotate on a threaded support (not shown) such that turning knob 116 manually one way pulls or pushes cable 106d against an internal wall of hub 102b and locks it in place relative to hub 102b and cannula 102 to thereby prevent cable 106b and grasper 118 from axial motion relative to cannula 102. Turning knob 116 the other way releases cable 106b and thus grasper 118 for axial motion relative to cannula 102.

When cannula 102 is being inserted into a patient, typically grasper 118 is in its retracted position shown in FIG. 5D, and typically is kept in that position until a practitioner has decided to grasp tissue or an object. A practitioner then pushes push-pull button 106c to move grasper distally from distal port 102d to open grasper jaws 172, 174 partway, to a relative position between the positions shown in FIGS. 5A and 5B, or all the way to the open position shown in FIG. 5A. After performing a desired medical procedure with grasper 118, a practitioner typically retracts grasper 118 and any tissue or object grasped thereby into lumen 110, to a position as shown in FIG. 5D or even further proximally and may extract cannula 102 from the patient.

According to some embodiments, cannula 108 is configured to rotate about axis A relative to hub 102b and thus relative to reusable position 104, as indicated by arrow B in FIGS. 1-3. A practitioner can control the rotation manually, by grasping cannula 108 and/or proximal port 112 and turning it relative to hub 102b, or by a manual lever or wheel mounted to hub 102b or to reusable portion 104, or with a motor in reusable portion 104 or in hub 102b controlled with a switch or joystick on reusable portion 104. The rotation can be though angles such as plus/minus 180 degrees or some other desired angle to enable visualizing an internal organ from some or all possible viewpoints. Mechanisms for cannula rotation are described in more detail below.

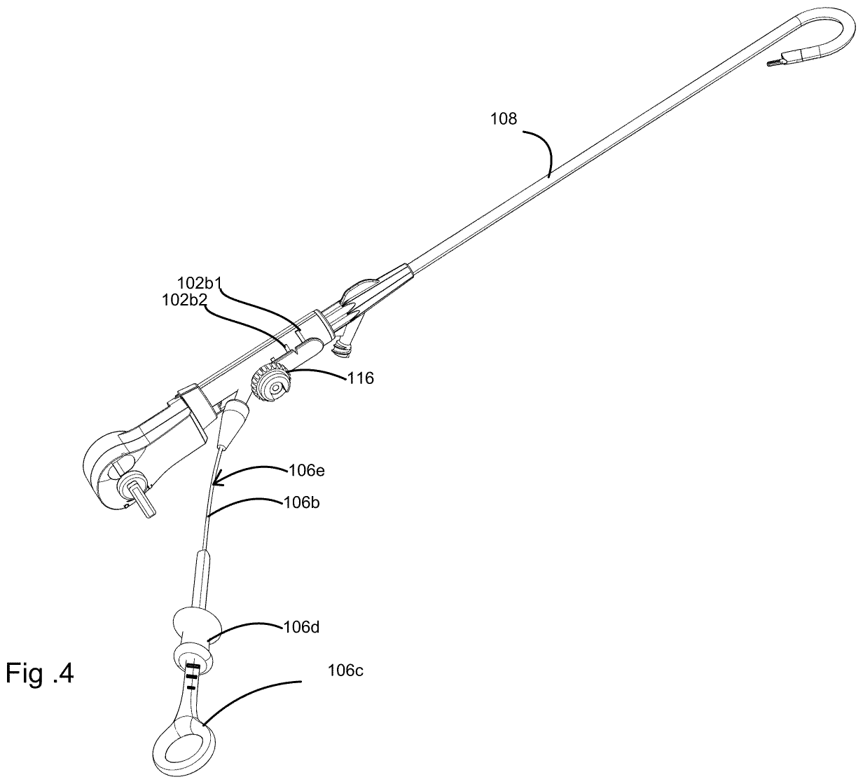

According to some embodiments, distal part 102c of cannula 102 is configured for deflection or angulation from axis A through angles such as up to 270 degrees or another desired angle range. FIGS. 1-4 show such angulation through an angle of roughly 270 degrees. Angulation through other angles, including no angulation, is not shown but should be understood as included in operation of endoscope 100. A practitioner can control such angulation completely manually, through a lever or wheel on reusable portion 104 or with an electric motor in reusable portion 104 or in hub 102b and a switch or joystick as described in more detail below.

Single-use portion 102 releasably attaches to reusable portion 104 as illustrated in FIG. 6 and as described in more detail in said parent application Ser. No. 18/083,209 which is incorporated by reference herein. FIG. 6 is like a portion of FIG. 1B of the parent application except for renumbering of the Figure and of some of the reference numerals to conform to the nomenclature used in this application.

FIG. 6 is an exploded perspective view of reusable portion 104 and proximal part 102a of cannula 102 and shows an example in which an electric motor rotates cannula 102 about cannula axis A relative to reusable portion 104 as directed by a practitioner manually operating electrical switches. Similar motor control can be used to control angulation of distal part 102c of cannula 102.

Reusable portion 104 comprises a pistol-grip handle 606 that extends along a handle axis D transverse to cannula axis A and is configured to be hand-held. Reusable portion 104 has an elongated slot 608 that has an open side facing to the right and extends along cannula axis A. Proximal part 102a of single-use portion 102 releasably attaches to reusable portion 104 at this slot 108. Reusable portion 104 comprises an internal power source 619 such as a battery and an internal motor 611 that are schematically illustrated. A gear shaft 613 extends into slot 608, along an axis C that is perpendicular to cannula axis A and handle axis D and is coupled with motor 611 to be selectively rotated thereby about axis C clockwise and counterclockwise as needed over selected angles of rotation. Motor 611 can be a step motor and can be directly behind gear shaft 613 such that the motor shaft rotates about an axis parallel to axis C. Gear shaft 613 is configured to engage and drive a shaft (not shown) extending to the right in proximal part 102a of single-use portion 102 when endoscope 100 is assembled. Handle 606 has at its distal face push buttons 615 and 617 that selectively couple power source 609 with motor 611 to robotically cause gear shaft 613 to rotate in a selected angular direction over a selected angle. For example, button 613 rotates cannula 102 counterclockwise relative to reusable portion 104 when pressed and button 617 rotates cannula 102 clockwise when pressed. Buttons 615, 617 preferably are positioned to be operated with the forefinger and/or middle finger of a user grasping handle 606.

Reusable portion 104 preferably includes a display 642 mounted thereon and operatively coupled with imaging module 330. Both preferably are powered with power source 619. In addition, reusable portion 104 has a button 644 at the proximal end thereof positioned for operation with the thumb of a user grasping handle 606. Button 644 is operatively coupled with imaging module 330 at the distal end of cannula 102 to control operations of imaging module 330 such as imaging functions and may be coupled with display 642 to control display functions. Button 644 can comprise two or more buttons or another suitable interface to control respective functions of imaging module 330 and/or of display 642. Reusable portion 104 further includes a pivoting latch 646 shown pivoted down to a closed position in FIG. 1 in which it latches to reusable portion 104 to hold reusable portion 104 and single-use portion 102 together. When pivoted up to an open position, latch 646 allows snapping single-use portion 102 into slot 608 of reusable portion 104.

Rotation of shaft 613 by motor 644 can be translated to rotation of cannula 102 using a power transfer mechanism as shown in FIGS. 1C and 2E of said parent application Ser. No. 18/083,209.

In addition to or instead of rotating cannula 108 relative to reusable portion 104 as described above, endoscope 100 can be configured to angulate distal part 102c of single-use portion 102 as described in said parent application Ser. No. 17/941,884 incorporated herein by reference, by using an electric motor that a practitioner controls with a touch pad or joystick schematically illustrated in FIG. 6, on the reusable portion of endoscope 100 (see also FIG. 1 in the parent patent), or by using a mechanical link 146 (FIG. 6) that a practitioner controls with a thumb lever (see lever 146 in FIG. 1B of said parent patent application Ser. No. 17/835,624) or with a wheel serving in place of lever 146.

In embodiments using an electric motor to angulate the cannula, an additional fail-safe control can be added to return the cannula to no angulation or some very low or minimal degree of angulation in case of failure of cannula 102 to return to that state due to, for example, power failure or electronic glitch. This control can be achieved by spring biasing distal part 102c to a no angulation state with sufficient force to return the cannula to a no angulation state but such that the motor can overcome the bias force when a practitioner engages the motor to angulate the cannula. One benefit of such control is to ensure that a cannula can always return to a no angulation or some low degree of angulation when a practitioner desires to extract a cannula from a patient and electric motor control over angulation fails or in otherwise insufficiently effective.

An alternative way of mating a single-use portion to a reusable portion to assemble an endoscope, which can be used to mate a reusable portion such as portion 104 described above to portion 102 described above to assemble endoscope 100 is disclosed in said parent application Ser. No. 15/855,532 incorporated herein by reference.

In some embodiments, endoscope 100 can be operatively coupled to an external processor and display unit 652 via a cable 650 or a wireless connection 654, as described in more detail in said patent application incorporated by reference. Endoscope 100 then can omit display 642 and some or all of the image processing electronics in reusable portion 104 and use instead image processing electronics and a display in unit 652. Alternatively, images from module 330 can be displayed both at display 642 and at unit 652, and each can be configured to send commands to module 330 and to manipulate the displayed images by using a touch screen and/or other interface facilities.

Preferably, single-use portion 102 is shipped to end-users sealed in a sterile pouch together with said surgical implement 106. The two can be in the same sterile pouch, with implement 106 assembled into single-use portion, or the two can be shipped in separate sterile envelopes and assembled when needed for a medical procedure by threading cable 106b and the grasper or other medical device at its distal end through port 114 and securing casing 106e to port 114 in case no casing 106e is used.

Although the foregoing has been described in some detail for purposes of clarity, it will be apparent that certain changes and modifications may be made without departing from the principles thereof. There can be many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A compact robotic endoscope comprising:
a reusable portion;
a single-use portion comprising (a) a proximal part configured to releasably couple to the reusable portion to form said endoscope, (b) a hub that extend distally from the proximal part along a cannula axis and has a hub port, (c) a cannula that extends distally from the hub along the cannula axis and has an imaging module and a distal port at a distal part thereof, and (d) an internal lumen that extends from the hub port to said distal port;
a surgical instrument comprising a push-pull assembly that includes a finger-hold, a push-pull button configured to move distally and proximally relative to the finger-hold, and a cable with a surgical device secured to a distal end thereof;
wherein a proximal end of said cable is secured to said push-pull button and the cable extends therefrom to and though said hub port and said lumen to said surgical device and is configured to move the surgical device distally and proximately relative to the cannula with motion of the push-pull button relative to the finger-hold;
a locking knob secured to an intermediate portion of the cable and configured to move distally and proximally relative to the hub with said cable and to selectively lock the cable to the hub to thereby lock the surgical device at a selected position relative to the cannula; and
readily discernable indicia structures on the hub and/or the push-pull button configured to indicate an axial position of said locking knob relative to the hub and/or of said push-pull button relative to the finger-hold and thus an axial position of said surgical device relative to said cannula.

2. The compact robotic endoscope of claim 1, in which said cannula is configured to rotate about a cannula axis relative to said reusable portion, and an electric motor operatively coupled with the cannula to rotate the cannula about said cannula axis through selected angles relative to the reusable portion and a manual control operatively coupled with the motor to selectively cause the motor to rotate the cannula through said angles.

3. The compact robotic endoscope of claim 1, in which said surgical device secured to the distal end of the cable comprises a grasper configured to move between a retracted position in the cannula and an extended position in which the grasper protrudes distally from the cannula.

4. The compact robotic endoscope of claim 3, in which said grasper is spring-biased to open with distal axial motion relative to the cannula and to close with proximal axial motion relative to the cannula.

5. The compact robotic endoscope of claim 1, in which said distal part of the cannula is configured to angulate and said reusable portion includes a manual control to cause the cannula to angulate through selected angles relative to the long dimension of the cannula.

6. The compact robotic endoscope of claim 1, in which said reusable portion comprises a slot extending along a cannula axis and configured to enable insertion therein of said proximal part of the single-use portion in a motion transverse to the cannula axis.

7. The compact robotic endoscope of claim 1, in which said cannula further includes a proximal fluid port coupled with said lumen and configured to selectively rotate with the cannula about said cannula axis relative to the reusable portion.

8. The compact robotic endoscope of claim 1, in which said hub port is valved to prevent fluid flow out of the hub port.

9. The compact robotic endoscope of claim 1, in which the surgical device secured to the distal end of the cable comprises a grasper having distal resilient jaws spring-biased away from each other and proximal arch-shaped portions configured to engage said cannula as the grasper moves proximally relative to the cannula and thereby overcome said spring-bias and bring the jaws closer to each other.

10. The compact robotic endoscope of claim 1, in which said single-use portion together with said surgical instrument is configured for shipment to an end user in a sterile package.

11. The compact robotic endoscope of claim 1, in which said surgical instrument is permanently built into said single-use portion.

12. The compact robotic endoscope of claim 1 in which said indicia are sufficiently large to be felt with a gloved finger.

13. An endoscope comprising:
a reusable portion and a single-use portion releasably secured to the reusable portion;
wherein the single-use portion comprises a proximal part configured to releasably couple to the reusable portion to form said endoscope, a hub extending distally from the proximal part along a cannula axis, a cannula extending distally from the hub, and an internal lumen;
wherein:
the cannula has at a distal part thereof an imaging module and a distal port communicating with said lumen;
the hub has a hub port communicating with said lumen; and
the single use portion further includes a surgical instrument comprising a manually operated control assembly secured to the hub port, a cable that passes through the hub port and the lumen, and a surgical device at a distal end of the cable and configured to move relative to the cannula between a distal position and a retracted position in response to manual operation of said control assembly; and
physical indicia on at least one of the hub and the control assembly moving relative to said hub and/or control assembly in a motion matching said motion of the surgical device relative to the cannula and providing an indication of the instantaneous position of the surgical device relative to the cannula.

14. The endoscope of claim 13, including a locking mechanism secured to at least one of the cables and the control assembly and configured to selectively lock said surgical device in a selected position relative to the cannula.

15. The endoscope of claim 14, in which said indicia comprise indentations of ridges on the hub and said locking mechanism comprises a knob secured to said cable and configured to move therewith relative to said indentations and thereby instantaneously indicate the position of the surgical device relative to the cannula.

16. The endoscope of claim 13 in which said manually operated control assembly comprises a finger-hold configured to maintain a set distance from the hub port and a push-pull element coupled to a proximal end of the cable and configured to move relative to said finger-hold, and said indicia comprise indentations of ridges on a the movable element that mover relative to the finger-hold to thereby indicate the position of the surgical device relative to the cannula.

17. An endoscopy method comprising:
introducing a cannula portion of a single-use portion of the endoscope into an interior space in a body;
taking images of the interior space with an imaging module at a tip of the cannula;
manually operating a control assembly built in the single-use portion to selectively move a cable that originates at the control assembly and passes through a proximal port in the single-use portion and a lumen therein and terminates distally in a surgical device that moves between a distal position in which at least a portion thereof protrudes from the cannula and a retracted position in the cannula; and
detecting the position of the surgical device relative to the cannula by detecting visible displacement of the cable or of an element secured thereto relative to physical indicia that are on said single-use portion and are proximal of the cannula.

18. The method of claim 17, in which the step of manually operating the control assembly comprises selectively moving a push-pull button relative to a finger-hold to thereby push or pull said cable axially along a length of said lumen and thereby move said surgical device distally or proximally relative to a distal end of the cannula.

19. The method of claim 17, in which the step of moving the surgical device relative to the cannula comprises moving a grasper that has distal jaws biased away from each other and configured to move away from each other as the grasper moves distally relative to the cannula and to move toward each other as the grasper moves proximally relative to the cannula.

20. The method of claim 17, further including selectively locking the surgical device in a selected position relative to the cannula using a locking knob coupled to the cable and configured to selectively lock the cable to the cannula.

21. The method of claim 20, in which the step of detecting the position of the surgical device relative to the cannula comprises detecting the position of said locking knob relative to said indicia on said single-use portion.
